(19) United States
(12) Patent Application Publication
(10) Pub. No.: US 2026/0176233 A1
(43) Pub. Date: Jun. 25, 2026

(54) METHOD FOR PREPARING VITAMIN D2 USING A MICROCHANNEL REACTOR

(71) Applicant: FORMOSA LABORATORIES, INC., Taoyuan City (TW)

(72) Inventors: Chung-Yen HUANG, Taoyuan City (TW); Yun HSU, Taoyuan City (TW)

(21) Appl. No.: 19/429,175

(22) Filed: Dec. 22, 2025

(30) Foreign Application Priority Data
Dec. 23, 2024 (TW) 113150227

(51) Int. Cl. C07C 401/00; B01D 3/10; B01J 19/00; B01J 19/12

(52) U.S. Cl. CPC C07C 401/00; B01D 3/10; B01J 19/0093; B01J 19/123; B01J 2219/00936; B01J 2219/0877; C07C 2601/14; C07C 2602/24

(57) ABSTRACT

The present disclosure provides a method for preparing vitamin D₂ using a microchannel reactor, including: (a) dissolving ergosterol in a first solvent to form a first reaction solution; (b) introducing the first reaction solution into the microchannel reactor, and the first reaction solution is irradiated with light source A and light source B in sequence as the first reaction solution passes through a first photoreaction zone and a second photoreaction zone; (c) collecting and concentrating the first reaction solution irradiated by the light source B; (d) optionally, mixing the first reaction solution of step (c) with a second solvent to form a second reaction solution; (e) heating the first reaction solution of step (c) or the second reaction solution of step (d) to obtain the vitamin D₂.

### CROSS-REFERENCE TO RELATED APPLICATIONS

This application claims priority to and benefits from Taiwan Patent Application No. 113150227, filed on Dec. 23, 2024, which is incorporated herein by reference.

### BACKGROUND

### 1. Technical Field

The present disclosure relates to a method for preparing vitamin D₂, especially to a method for preparing vitamin D₂ using a microchannel reactor.

### 2. Description of Associated Art

In current, commercially available vitamin D₂ products are generally produced by irradiation in a flowing cell with a mercury lamp. This production method has advantages of low technical difficulty, and high power output of the mercury lamp, etc. However, this production method also has several disadvantages, including the generation of a large amount of isomers (such as tachysterol and lumisterol, etc.), a low vitamin D₂ content, and the short lifetime of the mercury lamp, etc.

To address the above issues, LED microchannel irradiation devices have been developed in recent years. The devices employ advanced UV-LED with microchannel plate modules, taking advantage of the ability to select a specific wavelength to reduce the generation of isomers during irradiation and to increase the vitamin content. Further, LEDs have a lifespan of more than 10 times longer than that of the mercury lamp, thereby mitigating the recycling issues associated with discarded mercury lamps. However, optimal utilization of UV-LEDs and microchannel reactors, as well as process optimization to achieve the highest productivity of vitamin D₂, remains a common goal among vitamin D₂ manufacturers.

### SUMMARY

In view of the foregoing, the present disclosure provides a method for preparing vitamin D₂ using a microchannel reactor, comprising:
(a) dissolving ergosterol in a first solvent to form a first reaction solution;
(b) introducing the first reaction solution into the microchannel reactor defined with a first photoreaction zone irradiated by a first light source and a second photoreaction zone located downstream of the first photoreaction zone and irradiated by a second light source, the first reaction solution being irradiated with the first light source and the second light source in sequence as the first reaction solution passes through the first photoreaction zone and the second photoreaction zone, wherein the first light source has a wavelength of 260 nm to 300 nm, the second light source has a wavelength of 345 nm to 385 nm, and the first reaction solution is irradiated by the first light source and the second light source in the first photoreaction zone and the second photoreaction zone at an irradiation flux ratio from 0.5:1 to 25:1;
(c) collecting and concentrating the first reaction solution irradiated by the second light source;
(d) optionally, mixing the concentrated first reaction solution of step (c) with a second solvent to form a second reaction solution;
(e) heating the first reaction solution of step (c) or the second reaction solution of step (d) to obtain the vitamin D₂.

In an embodiment, the light source is an LED lamp or a mercury lamp.

In an embodiment, the first solvent comprises any solvent selected from the group consisting of methanol, ethanol, isopropanol, methyl t-butyl ether (MTBE), tetrahydrofuran (THF), hexane, heptane, ethyl acetate, and acetone, or a combination thereof.

In an embodiment, the second solvent comprises at least two selected from the group consisting of acetonitrile, methanol, ethanol, N-methylpyrrolidone, acetone, and tetrahydrofuran.

In an embodiment, the second solvent comprises acetonitrile and methanol, and the volume ratio of the acetonitrile to the methanol is 1:1 to 1:10.

In an embodiment, the concentrating of step (c) is removing the first solvent by vacuum distillation.

In an embodiment, ergosterol is present in the reaction solution at a concentration of 5 g/L to 20 g/L.

In an embodiment, the heating is at a temperature of 60° C. to 70° C.

In an embodiment, the heating is performed for 2 hours to 4 hours.

In an embodiment, further comprising, cooling the heated first reaction solution of step (c) or the second reaction solution of step (d) after heating the first reaction solution of step (c) or the second reaction solution of step (d); and filtering and concentrating the first reaction solution of step (c) or the second reaction solution of step (d) in sequence.

In an embodiment, the cooling is reducing a temperature to 0° C. to 5° C.

In an embodiment, the cooling is performed for 1 hour to 2 hours.

The present disclosure further provides a microchannel reactor used for preparing vitamin D₂. The microchannel reactor is defined with a first photoreaction zone and a second photoreaction zone located downstream of the first photoreaction zone, and comprises: an irradiator emitting UV light, including a first source disposed in the first photoreaction zone and a second light source disposed in the second photoreaction zone, wherein the first light source has a wavelength different from that of the second light source; and at least one microchannel extending from its upstream to downstream and disposed in the first photoreaction zone and the second photoreaction zone, allowing a first reaction solution with dissolved ergosterol to flow from the upstream to the downstream of the at least one microchannel, wherein the reaction solution is irradiated by the first light source and the second light source in the first reaction zone and the second reaction zone at an irradiation flux ratio of 0.5:1 to 25:1.

In an embodiment, the microchannel reactor further comprises an inlet in the first reaction zone and an outlet in the second reaction zone, and an upstream end and a downstream end of the at least one microchannel are fluidly connected to the inlet and outlet, respectively.

In an embodiment, the first light source and the second light source of the microchannel reactor are independently selected from LED lamps or mercury lamps.

In an embodiment, the first light source has a wavelength of 260 nm to 300 nm, and the second light source has a wavelength of 345 nm to 385 nm.

In the present disclosure, by combining light sources with wavelengths of 260 nm to 300 nm and 345 nm to 385 nm within a microchannel reactor, ergosterol is subjected to a sequentially integrated irradiation reaction to produce vitamin D₂. Compared to traditional or conventional manners, production of vitamin D₂ by the device and method provided in the present disclosure achieves the efficacies in reducing proportion of isomers generated by irradiation, increasing the yield of vitamin D₂, and decreasing the occurrence of degradation reactions, which represents an inventive and breakthrough development in the field of drug substance production.

### BRIEF DESCRIPTION OF THE DRAWINGS

FIG. 1 is a flowchart of preparing vitamin D₂ by using a microchannel reactor according to an embodiment of the present disclosure.

FIG. 2 is a schematic stereogram of the microchannel reactor used for preparing vitamin D₂ according to an embodiment of the present disclosure.

FIG. 3 is a flowchart of subjecting ergosterol to a photo-isomerization and thermal isomerization to yield vitamin D₂ according to an embodiment of the present disclosure.

### DETAILED DESCRIPTION OF THE PREFERRED EMBODIMENTS

The execution modes of the present disclosure are illustrated by particular embodiments, and a person having the ordinary skill in the technical field to which the present disclosure belongs can readily appreciate the scope and efficacy of the present disclosure based on the content recorded herein. However, the embodiments recorded herein are not intended to limit the present disclosure. The technical features or schemes listed can be combined with one another. The present disclosure can be implemented or applied by other different execution modes. Details recorded herein can be altered or modified differently according to different viewpoints and applications without departing from the present disclosure.

Unless stated otherwise, “comprising”, “containing” or “having” particular elements used herein means that other elements such as units, components, structures, regions, parts, devices, systems, steps and connection relationships can be also included rather than excluded.

Unless expressly stated otherwise, the singular forms “a”, “an” and “the” also include the plural forms, and the “or” and “and/or” can be used interchangeably herein.

The numerical ranges described herein are inclusive and combinable, and any value falling into the numerical ranges described herein can be used as the upper or lower limit to derive a subrange. For example, a numerical range of “0.1 minute to 10 minutes” should be understood to include any subranges between the endpoints 0.1 and 10, e.g., subranges of 0.1 to 9, 0.1 to 8, 0.1 to 7, 0.1 to 6, 0.1 to 5, 0.1 to 4, 0.1 to 3, 0.1 to 2, 0.1 to 1, 0.1 to 0.9, 0.2 to 10, 0.3 to 10, 0.4 to 10, 0.5 to 10, 0.6 to 10, 0.7 to 10, 0.8 to 10, 0.9 to 10. In addition, a value falling into one range described herein should be considered to be included in the range of the present disclosure, for example, “0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, or 10 minute(s)” falls into the range “0.1 minute to 10 minutes” of the present disclosure.

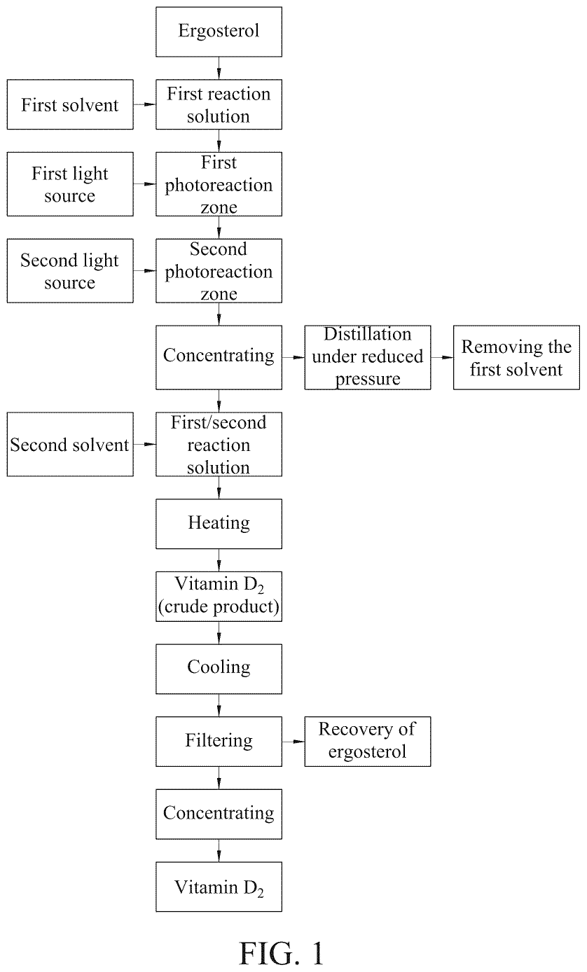

The inventors of the present disclosure found that, by preparing vitamin D₂ using a microchannel reactor, the proportion of isomers generated by irradiation can be decreased effectively, the yield of vitamin D₂ can be increased, and the occurrence of degradation reactions can be reduced. For example, referring to FIG. 1, the present disclosure provides a method for preparing vitamin D₂ using a microchannel reactor, comprising:
(a) dissolving ergosterol in a first solvent to form a first reaction solution;
(b) photo-chemical reaction: introducing the first reaction solution into the microchannel reactor which is defined with a first photoreaction zone irradiated by a first light source and a second photoreaction zone located downstream of the first photoreaction zone and irradiated by a second light source, so that the first reaction solution is irradiated with the first light source and the second light source in sequence as the first reaction solution passes through the first photoreaction zone and the second photoreaction zone, wherein the first light source has a wavelength of 260 nm to 300 nm (e.g., 280±10 nm or 280±5 nm, but not limited thereto), the second light source has a wavelength of 345 nm to 385 nm (e.g., 365±10 nm or 365±5 nm, but not limited thereto), and the first reaction solution is irradiated by the first light source and the second light source in the first photoreaction zone and the second photoreaction zone at an irradiation flux ratio of 0.5:1 to 25:1, e.g., 0.5:1 to 20:1, 0.5:1 to 15:1, 0.5:1 to 10:1, 0.5:1 to 5:1, 0.5:1 to 1:1, 0.6:1 to 25:1, 0.7:1 to 25:1, 0.8:1 to 25:1, 0.9:1 to 25:1, 1:1 to 25:1, 0.6:1 to 20:1, 0.7:1 to 15:1, 0.8:1 to 10:1, or 0.9:1 to 5:1, but not limited thereto;
(c) collecting and concentrating the first reaction solution irradiated by the second light source;
(d) optionally, mixing the concentrated first reaction solution of step (c) with a second solvent to form a second reaction solution;
(e) thermal isomerization: heating the first reaction solution of step (c) or the second reaction solution of step (d) to obtain the vitamin D₂ (crude product).

As used herein, the term “irradiation flux ratio” refers to “irradiation time X irradiation intensity”. In an embodiment, the irradiation time for the first reaction solution being irradiated by the first light source in the first photoreaction zone can be 0.2 minute to 2 minutes, e.g., 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2.0 minutes, and the irradiation time for the first reaction solution being irradiated by the second light source in the second photoreaction zone can be 0.2 to 0.5 minute, e.g., 0.2, 0.3, 0.4, or 0.5 minute, but not limited thereto. In another embodiment, the irradiation intensity of the first light source can be 50 mW/cm² to 250 mW/cm², e.g., 50, 60, 70, 80, 90, 100, 110, 120, 130, 140, 150, 160, 170, 180, 190, 200, 210, 220, 230, 240, or 250 mW/cm², and the irradiation intensity of the second light source can be 100 mW/cm² to 500 mW/cm², e.g., 100, 120, 140, 160, 180, 200, 220, 240, 260, 280, 300, 320, 340, 360, 380, 400, 420, 440, 460, 480, or 500 mW/cm², but not limited thereto. In the present disclosure, the irradiation time refers to the period during which the first reaction solution or photoreaction solution from the inlet to the outlet of the microchannel; the irradiation intensity (mW/cm²) refers to the intensity measured with an illuminometer at a distance of 1 cm from the light source.

In an embodiment, the first light source and the second light source are independently selected from LED lamps or mercury lamps. In another embodiment, the first light source and the second light source are preferably LED lamps.

In an embodiment, the first solvent comprises any one selected from the group consisting of methanol, ethanol, isopropanol, methyl t-butyl ether (MTBE), tetrahydrofuran (THF), hexane, heptane, ethyl acetate, and acetone, or a combination thereof. In another embodiment, the first solvent is preferably ethyl acetate.

In an embodiment, the second solvent comprises at least two selected from the group consisting of acetonitrile, methanol, ethanol, N-methylpyrrolidone, acetone, and tetrahydrofuran. In another embodiment, the second solvent preferably comprises acetonitrile and methanol. In still another embodiment, the volume ratio of acetonitrile to methanol can be 1:1 to 1:10, e.g., can be 1:1 to 1:2, 1:1 to 1:3, 1:1 to 1:4, 1:1 to 1:5, 1:1 to 1:6, 1:1 to 1:7, 1:1 to 1:8, 1:1 to 1:9, or 1:1 to 1:10. In an embodiment, the second solvent has a composition different from that of the first solvent. In an embodiment, at least on solvent of the second solvent composition has a boiling point higher than that of the first solvent.

In an embodiment, the concentrating of step (c) is removing the first solvent by vacuum distillation; and the mixing of step (d) is optionally adding the second solvent in batches. In the present disclosure, since the second solvent consists of at least two solvents, in an embodiment, the second solvent can be prepared and then mixed with the concentrated product of step (c) to form the second reaction solution; alternatively, in another embodiment, optionally, adding one solvent (such as acetonitrile) to mix with the concentrated product of step (c), and then adding the other solvent (such methanol) to form the second reaction solution, in batches, but not limited thereto.

In an embodiment, the ergosterol can be present in the first reaction solution at a concentration of 5 g/L to 20 g/L, e.g., can be 5, 6, 7, 8, 9, 10, 11, 12, 13, 14, 15, 16, 17, 18, 19, or 20 g/L.

In an embodiment, the heating can be performed at a temperature of 60° C. to 70° C., e.g., at 60, 61, 62, 63, 64, 65, 66, 67, 68, 69, or 70° C.

In an embodiment, the heating can be performed for 2 hours to 4 hours, e.g., for 2, 3, or 4 hours.

In the present disclosure, after the photo-chemical reaction and thermal isomerization are completed, purification can be further performed on the reactants to recover remained ergosterol and purify vitamin D₂. Therefore, as shown in FIG. 1, in an embodiment, further comprising, after heating the first reaction solution of step (c) or the second reaction solution of step (d), cooling the heated first reaction solution of step (c) or the second reaction solution of step (d); and filtering and concentrating the first reaction solution of step (c) or the second reaction solution of step (d) in sequence, to recover ergosterol and purify vitamin D₂.

In an embodiment, the cooling is dropping the temperature to 0° C. to 5° C., e.g., to 0, 1, 2, 3, 4, or 5° C.

In an embodiment, the cooling is performed for 1 hour to 2 hours, e.g., for 1, 1.5, or 2 hour(s).

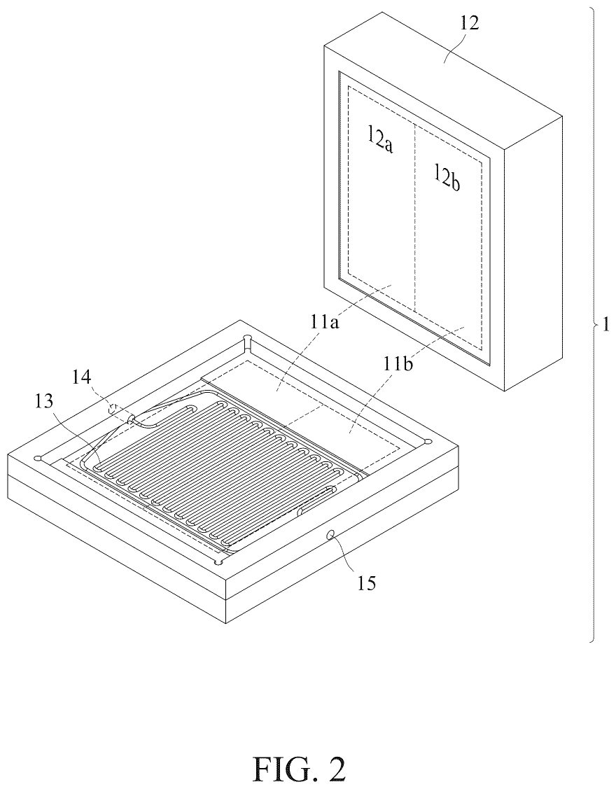

In addition, in order to achieve the efficacies of low proportion of isomers generated and high yield of vitamin D₂ in the present disclosure, as shown in FIG. 2, the present disclosure further provides a microchannel reactor 1 used for preparing vitamin D₂, which is defined with a first photoreaction zone 11a and a second photoreaction zone 11b located downstream of the first photoreaction zone 11a, and the microchannel reactor 1 comprises: an irradiator 12 emitting UV light, including a first source 12a disposed in the first photoreaction zone 11a and a second light source 12b disposed in the second photoreaction zone 11b, and the first light source 12a has a wavelength different from that of the second light source 12b; and at least one microchannel 13, disposed by extending from its upstream to downstream in the first photoreaction zone 11a and the second photoreaction zone 11b, for a first reaction solution with dissolved ergosterol flowing from the upstream to the downstream of the at least one microchannel 13, wherein the reaction solution is irradiated by the first light source 12a and the second light source 12b in the first reaction zone 11a and the second reaction zone 11b at an irradiation flux ratio of 0.5:1 to 25:1. In addition, a housing upper lid where the irradiator 12 located covers over at least one microchannel 13 during reaction.

In an embodiment, the microchannel 13 within the microchannel reactor 1 has the following dimensions: depth of 1 mm to 2 mm, e.g., 1, 1.1, 1.2, 1.3, 1.4, 1.5, 1.6, 1.7, 1.8, 1.9, or 2 mm; width of 1 mm to 3 mm, e.g., 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 mm.

In an embodiment, the microchannel reactor further comprises an inlet 14 in the first reaction zone 11a and an outlet 15 in the second reaction zone 11b, and the at least one microchannel 13 has an upstream end and a downstream end in fluid communication with the inlet 14 and outlet 15, respectively.

In an embodiment, the first light source 12a and the second light source 12b of the microchannel reactor are independently selected from LED lamps or mercury lamps. In another embodiment, the first light source 12a and the second light source 12b are preferably LED lamps.

In an embodiment, the first light source 12a has a wavelength of 260 nm to 300 nm, and the second light source 12b has a wavelength of 345 nm to 85 nm. In another embodiment, the first light source 12a has a wavelength of 280±10 nm or 280±5 nm, but not limited thereto; in still another embodiment, the second light source 12b has a wavelength of 365±10 nm or 365±5 nm, but not limited thereto.

In an embodiment, when the irradiator 12 covers over the microchannel 13 for irradiating, the first light source 12a and/or the second light source 12b can be located from the microchannel 13 at a distance of 1 cm to 3 cm, e.g., 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 cm, but not limited thereto.

In the present disclosure, the microchannel reactor can be in the form of “one” or “a set”, i.e., can have one microchannel or a plurality of microchannels as a set. In particular, a microchannel reactor is within the scope claimed by the present disclosure, as long as the microchannel reactor allowed a first reaction solution to pass into photoreaction zones wherein the first reaction solution is irradiated by a first light source with a wavelength of 260 nm to 300 nm (i.e., the first photoreaction zone defined in the present disclosure) and is irradiated by a second light source with a wavelength of 345 nm to 385 nm (i.e., the second photoreaction zone defined in the present disclosure), and the first reaction solution is irradiated by the first light source and the second light source in the first photoreaction zone and the second photoreaction zone at an irradiation flux ratio of 0.5:1 to 25:1.

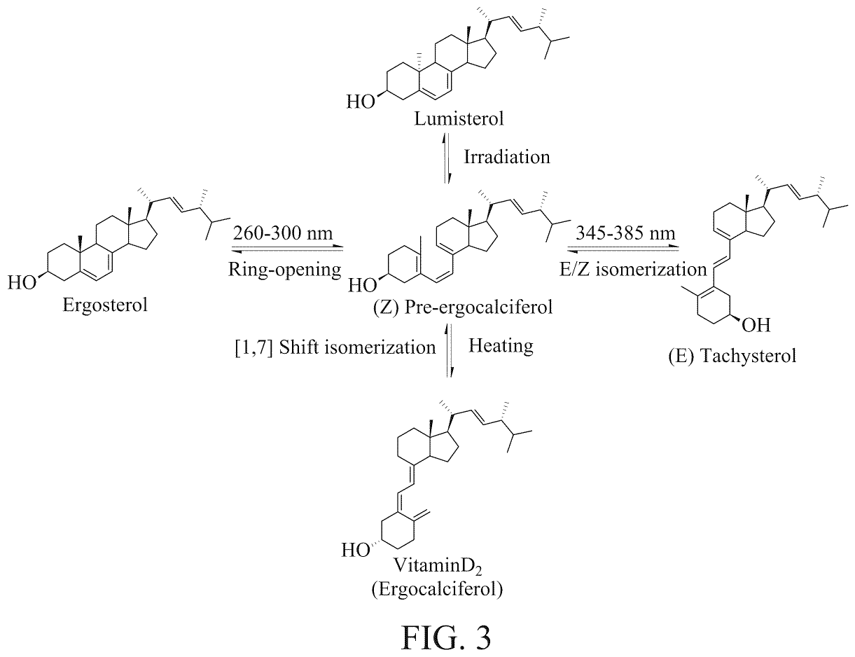

In the present disclosure, as shown in FIG. 3, ergosterol is irradiated by a light source having a wavelength of 260 nm to 300 nm to perform a ring-opening reaction, thereby being converted to pre-ergocalciferol, which is subjected to photo-isomerization by utilizing a light source having a wavelength of 345 nm to 385 nm, to convert the isomer tachysterol into pre-ergocalciferol, thereby increasing the content of pre-ergocalciferol. Finally, thermal isomerization is performed to synthesize vitamin D₂ (ergocalciferol). Accordingly, by preparing vitamin D₂ using the method and the microchannel reactor developed in the present disclosure, the proportion of isomers (such as lumisterol and tachysterol) generated by irradiation is decreased effectively, the yield of vitamin D₂ is increased, and the occurrence of degradation reactions is reduced.

The present disclosure illustrates the details through Examples. However, the interpretation of the present disclosure shouldn't be considered as limiting that of the following examples.

### Example 1

This example describes a method for preparing vitamin D₂, including the following steps:
A. Photo-chemical reaction (photo-ring-opening and photo-isomerization reaction): ergosterol (20 g), 1% (0.2 g) 9-acetylanthracene (9-AAA), 75V (1500 ml) ethyl acetate (EtOAc), and 0.25% (0.05 g) 2,6-dibutylhydroxytoluene (BHT) were charged into a reaction flask, and stirred at room temperature (20° C. to 30° C.) until completely dissolved, thereby forming a photoreaction solution; LED lamps with wavelengths of 280 nm and 365 nm were then switched on, with irradiation intensities of 180 mW/cm² and 250 mW/cm², respectively. The photoreaction solution was subsequently injected into a microchannel reactor (with a microchannel size of 1 mm in depth and 2 mm in width) and irradiated sequentially by LED lamps at 280 nm and 365 nm wavelengths for 0.8 minute and 0.2 minute, respectively. The distance from the light source to the microchannel was about 2 cm. The photo-ring-opening and photo-isomerization reaction were thus completed in one step;
B. Concentrating the reaction solution: the irradiated solution was concentrated by vacuum distillation; then acetonitrile (ACN) was added to a volume of 2V (40 ml), followed by 10V (200 ml) methanol (MeOH), to formulate a thermal isomerization reaction solution;
C. Thermal isomerization reaction: the thermal isomerization reaction solution was warmed by heating for refluxing at 65° C. and allowed to perform thermal isomerization reaction for 4 hours, thereby obtaining a reaction solution containing vitamin D₂ (vitamin D₂ crude product).
D. Purification of materials: heating was stopped, the reaction solution containing vitamin D₂ was naturally cooled to room temperature for about 1 hour to 2 hours and continuously cooled to 0° C. to 5° C. for about 1 hour to 2 hours; solid was filtered and dried to obtain recovered ergosterol 5.8 g; and the filtrate was concentrated and dried to obtain crude vitamin D₂ product 13.8 g (vitamin D₂ amount of 9.0 g).

### Example 2

Example 2 employed the same raw materials and microchannel as those in Example 1 for preparing vitamin D₂, except that the ergosterol was fed at an amount of 10 g and irradiated by LED lamps with wavelengths of 280 nm and 365 nm both for 0.2 minute, to obtain recovered ergosterol 7.0 g; and the filtrate was concentrated and dried to obtain crude vitamin D₂ product 3.0 g (vitamin D₂ amount of 1.6 g).

### Example 3

Example 3 employed the same raw materials and microchannel as those in Example 1 for preparing vitamin D₂, except that the ergosterol was fed at an amount of 10 g and irradiated by LED lamps with wavelengths of 280 nm and 365 nm for 2 minute and 0.2 minute, respectively, to obtain recovered ergosterol 1.5 g; and the filtrate was concentrated and dried to obtain crude vitamin D₂ product 8.2 g (vitamin D₂ amount of 5.1 g).

### Comparative Example 1

Vitamin D₂ was prepared employing the raw materials as those in Example 1, except that the ergosterol was fed at an amount of 30 g, a flow cell mercury lamp reactor (Peschl Ultraviolet GmbH P-EVG 10) was employed to perform the photo-chemical reaction, and the irradiation was performed across all UV full wavelengths for 45 minute, to obtain recovered ergosterol 6.2 g; and the filtrate was concentrated and dried to obtain crude vitamin D₂ product 10.7 g (vitamin D₂ amount of 4.6 g).

### Comparative Example 2

Vitamin D₂ was prepared employing the raw materials as those in Example 1, except that the ergosterol was fed at an amount of 600 g, a batch cell-type mercury lamp reactor (AA-JIN INDUSTRY CO., LTD., PR-D02) was employed to perform the photo-chemical reaction, and the irradiation was performed across all UV wavelengths for 60 minute, to obtain recovered ergosterol 488.6 g; and the filtrate was concentrated and dried to obtain crude vitamin D₂ product 124.7 g (vitamin D₂ amount of 45.5 g).

### Experimental Example 1: Purity Assay

HPLC (Waters, Acquity UPLC H class) was employed to analyze the amounts of isomers and vitamin D₂ in Examples 1, 2, and 3 and Comparative Examples 1 and 2, wherein the isomer amount referred to the amounts of lumisterol and tachysterol after completion of the photo-chemical reaction (photo-ring-opening and photo-isomerization reaction), and the vitamin D₂ amount referred to the amount of the crude vitamin D₂ product after completion of the photo-chemical reaction and thermal isomerization reaction. The assay method and conditions for HPLC were shown in Table 1.

TABLE 1

| | |
|---|---|
| Column | Thermo hypurity C18 250*4.6 mm |
| Mobile phase | MeOH/ACN = 1/9 |
| Flow rate (mL/min) | 1 |
| Detection (UV, nm) | UV 254 nm |
| Running Time (min) | 40 |
| Column oven (° C.) | 30 |
| Pre-vitamin D₂ | RRT 0.93 |
| Tachysterol | RRT 0.96 |
| Vitamin D₂ | RRT 1.00 |
| Lumisterol | RRT 1.09 |
| Ergosterol | RRT 1.62 |

The results were shown in Table 2 below. It is clear that by preparing vitamin D₂ with the microchannel reactor of the present disclosure (Examples 1, 2 and 3), the ratio of isomers generated by irradiation was effectively improved and the yield of vitamin D₂ was increased, i.e., the purity and production efficiency of vitamin D₂ were enhanced. Irradiation Flux=Irradiation Time X Irradiation Intensity.

TABLE 2

| | | 280 nm/ | | | | Weight | | |
|---|---|---|---|---|---|---|---|---|
| | 280 nm/ | 365 nm | | | | of the | | |
| | 365 nm | Irradiation | Irradiation | | | crude | | |
| | Irradiation | Intensity | Flux Ratio | Isomers Content (%) | | vitamin | Vitamin D₂ | Yield |
| | Time (min) | (mW/cm²) | (280 nm:365 nm) | Tachysterol | Lumisterol | D₂ (g) | content (g) | (%) |
| Ex. 1 | 0.8/0.2 | 180/250 | 2.88:1 | 0.5 | 0.9 | 13.8 | 9.0 | 64.8 |
| Ex. 2 | 0.2/0.2 | 180/250 | 0.72:1 | 1.4 | 0.7 | 3.0 | 1.6 | 53.3 |
| Ex. 3 | 2/0.2 | 180/250 | 7.2:1 | 0.9 | 0.8 | 8.2 | 5.1 | 62.2 |
| Comp. | 45 | N/A | N/A | 1.3 | 3.7 | 10.7 | 4.6 | 43.4 |
| Ex. 1 | | | | | | | | |
| Comp. | 60 | N/A | N/A | 5.8 | 0.3 | 124.7 | 45.5 | 36.5 |
| Ex. 2 | | | | | | | | |

The above Examples are used for illustrating the principles and efficacies of the present disclosure only but not for limiting the present disclosure. Modifications can be made to above Examples by anyone skilled in the art without departing from the spirit and scope of the present disclosure. Therefore, the range claimed by the present disclosure should be as the claims attached.

What is claimed is:

1. A method for preparing vitamin D₂ using a microchannel reactor, comprising:
(a) dissolving ergosterol in a first solvent to form a first reaction solution;
(b) introducing the first reaction solution into the microchannel reactor defined with a first photoreaction zone irradiated by a first light source and a second photoreaction zone located downstream of the first photoreaction zone and irradiated by a second light source, the first reaction solution being irradiated with the first light source and the second light source in sequence as the first reaction solution passes through the first photoreaction zone and the second photoreaction zone, wherein the first light source has a wavelength of 260 nm to 300 nm, the second light source has a wavelength of 345 nm to 385 nm, and the first reaction solution is irradiated by the first light source and the second light source in the first photoreaction zone and the second photoreaction zone at an irradiation flux ratio from 0.5:1 to 25:1;
(c) collecting and concentrating the first reaction solution irradiated by the second light source;
(d) optionally, mixing the concentrated first reaction solution of step (c) with a second solvent to form a second reaction solution;
(e) heating the first reaction solution of step (c) or the second reaction solution of step (d) to obtain the vitamin D₂.

2. The method of claim 1, wherein the first light source and the second light source are independently selected from LED lamps or mercury lamps.

3. The method of claim 1, wherein the first solvent comprises any solvent selected from the group consisting of methanol, ethanol, isopropanol, methyl t-butyl ether, tetrahydrofuran, hexane, heptane, ethyl acetate, and acetone, or a combination thereof.

4. The method of claim 1, wherein the second solvent comprises at least two selected from the group consisting of acetonitrile, methanol, ethanol, N-methylpyrrolidone, acetone, and tetrahydrofuran.

5. The method of claim 1, wherein the second solvent comprises acetonitrile and methanol, and the volume ratio of the acetonitrile to the methanol is 1:1 to 1:10.

6. The method of claim 1, wherein the concentrating of step (c) is removing the first solvent by vacuum distillation.

7. The method of claim 1, wherein the ergosterol is present in the first reaction solution at a concentration of 5 g/L to 20 g/L.

8. The method of claim 1, wherein the heating is at a temperature of 60° C. to 70° C.

9. The method of claim 1, further comprising cooling the heated first reaction solution of step (c) or the second reaction solution of step (d) after heating the first reaction solution of step (c) or the second reaction solution of step (d); and filtering and concentrating the first reaction solution of step (c) or the second reaction solution of step (d) in sequence.

10. The method of claim 9, wherein the cooling is reducing a temperature to 0° C. to 5° C.

11. A microchannel reactor used for preparing vitamin D₂, which is defined with a first photoreaction zone and a second photoreaction zone located downstream of the first photoreaction zone, the microchannel reactor comprising:
an irradiator emitting UV light, including a first source disposed in the first photoreaction zone and a second light source disposed in the second photoreaction zone, and the first light source has a wavelength different from that of the second light source; and
at least one microchannel, extending from its upstream to downstream, disposed in the first photoreaction zone and the second photoreaction zone, allowing a first reaction solution with dissolved ergosterol to flow from the upstream to the downstream of the at least one microchannel, wherein the reaction solution is irradiated by the first light source and the second light source in the first reaction zone and the second reaction zone at an irradiation flux ratio of 0.5:1 to 25:1.

12. The microchannel reactor of claim 11, further comprising an inlet in the first reaction zone and an outlet in the second reaction zone, and an upstream end and a downstream end of the at least one microchannel being fluidly connected to the inlet and outlet, respectively.

13. The microchannel reactor of claim 11, wherein the first light source and the second light source are independently selected from LED lamps or mercury lamps.

14. The microchannel reactor of claim 11, wherein the first light source has a wavelength of 260 nm to 300 nm, and the second light source has a wavelength of 345 nm to 385 nm.
